(19) United States
(12) Patent Application Publication
(10) Pub. No.: US 2010/0185013 A1
(43) Pub. Date: Jul. 22, 2010

(54) PROCESS FOR FORMING ALPHA, BETA-UNSATURATED CARBONYL HALIDES

(76) Inventors: Matthew J. Pinnow, Woodbury, MN (US); Eric R. Choban, Williamstown, NJ (US); Kim M. Vogel, Lake Elmo, MN (US)

Correspondence Address: 3M INNOVATIVE PROPERTIES COMPANY, PO BOX 33427, ST. PAUL, MN, 55133-3427, US

(21) Appl. No.: 12/665,169

(22) PCT Filed: Jun. 4, 2008
(86) PCT No.: PCT/US08/65778

§ 371 Date: Dec. 17, 2009

Related U.S. Application Data
(60) Provisional application No. 60/946,763, filed on Jun. 28, 2007.

(51) Int. Cl. C07C 51/60

(52) U.S. Cl. 562/861

(57) ABSTRACT

A process for forming an α,β-unsaturated carbonyl halide in a microreactor is described. The reactants comprise an α,β-unsaturated carboxylic acid, a halogenating agent, and a catalyst. A first inlet stream, a second inlet stream, and an optional third inlet stream flow into a reaction chamber of a flow-through microreactor to form a reaction product comprising an α,β-unsaturated carbonyl halide.

### FIELD

The present disclosure relates to processes for forming α,β-unsaturated carbonyl halides in a flow-through microreactor.

### BACKGROUND

Halogenation of organic acids to form organic acid halides can produce valuable intermediates as described in U.S. Pat. No. 2,013,988 (Meder et al.). These intermediates may be used for further synthetic modification, for example, in fine chemical and pharmaceutical applications.

Microreactors can be used as tools for carrying out chemical reactions, and may have certain critical features in the millimeter or sub-millimeter size range. Microreactor technology represents a scaled-down version from that of conventional chemical reactors, either in the laboratory or in industry, where the typical feature sizes range from a few centimeters to several meters.

Microreactors can provide improved control of potentially hazardous chemical reactions. Greater control over such reactions may lead to improved safety and handling, and isolation of valuable reactive intermediate products. Microreactors can provide enhanced control over reagent mixing, fluid flow, and heat sinking/sourcing for chemical reactions as described in WO 99/22857 (Harston et al.).

### SUMMARY

The present disclosure describes processes for forming an α,β-unsaturated carbonyl halide. A process for forming α,β-unsaturated carbonyl halides in a microreactor is described. The reactants include an α,β-unsaturated carboxylic acid, a halogenating agent, and a catalyst. The reactants flow into a reaction chamber of a flow-through microreactor to form a reaction product, that being the α,β-unsaturated carbonyl halide.

In a first aspect, a process is provided for forming an α,β-unsaturated carbonyl halide. The process includes providing reactants for a solventless reaction. The reactants comprise an α,β-unsaturated carboxylic acid provided in a first inlet stream, a halogenating agent in a second inlet stream, and a catalyst in the first inlet stream or in an optional third inlet stream. The first inlet stream, the second inlet stream, and the optional third inlet stream are introduced into a reaction chamber of a flow-through microreactor. The process further includes forming a reaction product comprising an α,β-unsaturated carbonyl halide.

In a second aspect, a process is provided for forming an α,β-unsaturated carbonyl halide. The reactants for the process comprise an α,β-unsaturated carboxylic acid provided in a first inlet stream, a halogenating agent comprising oxalyl chloride in a second inlet stream, and a catalyst in the first inlet stream or in an optional third inlet stream. The first inlet stream, the second inlet stream, and the optional third inlet stream are introduced into a reaction chamber of a flow-through microreactor. The process further includes forming a reaction product comprising an α,β-unsaturated carbonyl halide, such that the α,β-unsaturated carbonyl halide has a percent yield of greater than 90 percent.

### BRIEF DESCRIPTION OF THE DRAWINGS

FIG. 1 is a schematic representation of a cross-section view of an exemplary flow-through microreactor.

FIG. 2 is a schematic representation of a top view of an exemplary flow-through microreactor.

FIG. 3 is a schematic representation of a top view of an exemplary reaction chamber of a flow-through microreactor.

FIG. 4 is a schematic representation of a top view of an exemplary flow-through microreactor having serpentine inlet channels.

### DETAILED DESCRIPTION

For the following defined terms, these definitions shall be applied, unless a different definition is given in the claims or elsewhere in the specification.

The term “solventless reaction” refers to a reaction substantially free of a solvent. The reaction typically contains less than 2 weight percent, less than 1 weight percent, less than 0.5 weight percent, less than 0.2 weight percent, or less than 0.1 weight percent of a solvent. Any solvent may be present that is not added intentionally, but is present as an impurity in the reaction.

The term “inlet stream” refers to reactants flowing from an entry location to a reaction chamber of a microreactor.

The term “reaction chamber” refers to a region or area where separate incoming streams contact one another. The reactants of the inlet streams mix with one another, where one or the other of the reactants may surround the other.

The term “flow-through microreactor” refers to a reactor having volume of up to 100 ml, up to 75 ml, up to 50 ml, up to 35 ml, up to 25 ml, up to 10 ml, or up to 1 ml. The reactor can have a volume of at least 0.01 ml, of at least 0.1 ml, of at least 1 ml, of at least 3 ml, or of at least 5 ml. The reactor can have a segmented or continuous flow of reactants flowing into a reaction chamber as well as a segmented or continuous flow of reaction products exiting the reaction chamber.

The term “alkyl” refers to a straight chain or branched C₁-C₂₀ hydrocarbon or a cyclic C₃-C₂₀ hydrocarbon.

The term “aryl” refers to aromatic groups such as phenyl. Aryl groups may also include fused polycyclic aromatic ring systems. The aryl often has 6 to 20 carbon atoms.

The term “percent yield” refers to the percent conversion of an organic acid to an organic halide. More particularly, percent yield refers to the percent conversion of an α,β-unsaturated carboxylic acid to an α,β-unsaturated carbonyl halide.

The term “solvent” refers to a chemical that is not a reactant or a catalyst.

The recitation of numerical ranges by endpoints includes all numbers subsumed within that range (e.g., 1 to 5 includes 1, 1.5, 2, 2.75, 3, 3.8, 4, and 5).

As included in this specification and the appended claims, the singular forms “a”, “an”, and “the” include plural referents unless the content clearly dictates otherwise. Thus, for example, reference to a composition containing “a compound” includes a mixture of two or more compounds. As used in this specification and appended claims, the term “or” is generally employed in its sense including “and/or” unless the content clearly dictates otherwise.

Unless otherwise indicated, all numbers expressing quantities or ingredients, measurement of properties and so forth used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings of the present disclosure. At the very least, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains errors necessarily resulting from the standard deviations found in their respective testing measurements.

The present disclosure describes a process for forming an α,β-unsaturated carbonyl halide in a flow-through microreactor. In many embodiments, the reaction is solventless. The reactants comprise an α,β-unsaturated carboxylic acid in a first inlet stream, a halogenating agent in a second inlet stream, and a catalyst in the first inlet stream or in an optional third inlet stream. The first inlet stream, the second inlet stream and the optional third inlet stream flow into a reaction chamber of a flow-through microreactor. The reactants contact one another in the reaction chamber. Contact of the reactants initiates the formation of the α,β-unsaturated carbonyl halide. As the α,β-unsaturated carbonyl halide is formed, a gas may be evolved. The gas provides chaotic mixing within the reaction chamber, such that the reaction chamber can be free of static mixing or mechanical mixing means. A reaction product comprising the α,β-unsaturated carbonyl halide is formed which exits the flow-through microreactor. The reaction product of the catalyzed chemical reaction is synthesized continuously or semi-continuously at an increased rate relative to an uncatalyzed chemical reaction at the same temperature. Using the flow-through microreactor, the α,β-unsaturated carbonyl halide can be formed having an increased percent yield, and a lower percent of a Michael addition product when with a catalyst in comparison to a process for forming an α,β-unsaturated carbonyl halide without a catalyst. A lower percent of an acid addition product (i.e., acid addition to the olefin group) is formed by the method described.

An α,β-unsaturated carbonyl halide can be formed for use as a chemical intermediate. The chemical intermediate can be used as a reactant for synthesizing additional molecules applicable for use in adhesives, coatings, petroleum applications, consumer applications, and industrial applications.

Methods of halogenating α,β-unsaturated carboxylic acids to form α,β-unsaturated carbonyl halides are known. For example, some methods are described in March, J., Advanced Organic Chemistry, 4^{th} Ed., John Wiley & Sons, p. 437-438 (1992). Various approaches have been used to control the reaction, which can be quite exothermic. For example, one approach is to use solvents to help control the reaction. That is, the reactants are diluted with solvent so that the reaction is not too exothermic. The reactions can occur in the presence or in the absence of a catalyst. If a catalyst is used, a solvent is typically needed to control the reaction. If a catalyst is not used, the reaction can be solventless. However, if a catalyst is not used, larger amounts of undesirable byproducts including Michael addition products and acid addition products tend to form. Most of the methods known for halogenating α,β-unsaturated carboxylic acids to form α,β-unsaturated carbonyl halides are in batch reactors. To isolate the reaction product, the solvent often must be removed and purification steps are often necessary to remove various byproducts.

In contrast to the known methods, a method is provided for forming α,β-unsaturated carbonyl halides in a flow-through microreactor. The reactants include an α,β-unsaturated carboxylic acid, a halogenating agent, and a catalyst. In many embodiments, the reaction is solventless. The α,β-unsaturated carbonyl halides can be produced continuously or semi-continuously at temperatures comparable to those used for uncatalyzed batch reactions. The percent yield of the α,β-unsaturated carbonyl halides can be higher and the byproducts can be lower than can be obtained using uncatalyzed batch reactions. Further, unlike batch reactions, both a catalyst and solventless conditions can be used to produce a high purity, high yield reaction product. A solventless reaction mixture can be desirable in some applications. The lack of a solvent in the process can remove a down-stream processing step, and facilitate more efficient manufacture of the α,β-unsaturated carbonyl halide.

Halogenation of an α,β-unsaturated carboxylic acid may be accomplished in a flow-through microreactor. In some embodiments, the halogenation of the α,β-unsaturated carboxylic acid in the presence of a catalyst may occur using a solventless reaction. The reaction of an α,β-unsaturated carboxylic acid with a halogenating agent in the presence of a catalyst to yield an α,β-unsaturated carbonyl halide as a reaction product may be referred to as halo-de-hydroxylation. The chemical reaction can be described in Reaction A.

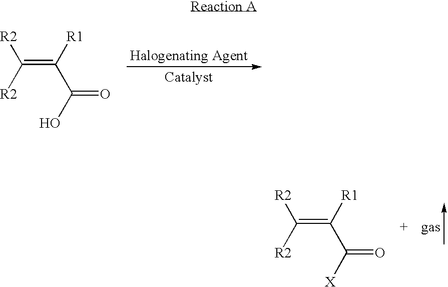

The reactants contact one another in a reaction chamber. The reactants may be a gas, or a liquid having sufficient viscosity, such that the liquid may be delivered with a metering pump for flowing into the flow-through microreactor. Some reactants may become a fluid or have sufficiently lower viscosity upon heating. The reactants may be solid at room temperature, and can be provided to a flow-through microreactor at a temperature above a melting point.

A first inlet stream may comprise an α,β-unsaturated carboxylic acid. The α,β-unsaturated carboxylic acid has a carbon-carbon double bond and a carbon-oxygen double bond (e.g., carbonyl group) that are separated by a single carbon-carbon single bond. The double bonds are conjugated, where the carbon-oxygen double bond (carbonyl group) of a carboxylic acid functional group is proximate to the carbon-carbon double bond of the olefinic functional group. The two functional groups provide reactive sites for further synthetic modification. For example, the carbonyl group of the α,β-unsaturated carboxylic acid may be converted in the presence of a halogenating agent to yield an α,β-unsaturated carbonyl halide.

The carboxylic acid of the α,β-unsaturated carboxylic acid may be ionizable, such that an anion of the carboxylic acid may react with another α,β-unsaturated carboxylic acid to yield a byproduct, for example, a Michael addition product. The Michael addition product may result from the nucleophilic addition of carbanions of α,β-unsaturated carboxylic acid compounds. The Michael addition reaction may be further described in Morrison, R. T. and Boyd, R. N., “The Michael Addition”, Organic Chemistry, 6^{th} Ed., Prentice Hall, Inc., 979-981 (1992).

The Michael addition product may be one of several byproducts that may form during the halogenation of an α,β-unsaturated carboxylic acid. Similarly, the acid addition product may be one of the byproducts formed during the halogenation of the α,β-unsaturated carboxylic acid. The formation or absence of byproducts in the halogenation of the α,β-unsaturated carboxylic acid may result from a number of factors. Some of the factors may include, but are not limited to, the selection of reactants, catalyst, temperature, residence time, impurities, solvent, presence of moisture, mixing, or combinations thereof.

The α,β-unsaturated carboxylic acids used in the first inlet stream are typically of Formula I. In Formula I, R1 may comprise hydrogen, substituted alkyl, unsubstituted alkyl, aryl, substituted aryl, or combinations thereof. Each R2 may independently comprise hydrogen, substituted alkyl, unsubstituted alkyl, unsubstituted aryl, substituted aryl, carboxyl, or combinations thereof. Suitable substituents for alkyl groups include, but are not limited to, carboxyl, alkoxyl, aryl, aryloxy or combinations thereof. Suitable substituents for aryl groups include, but are not limited to, alkyl, carboxyl, alkoxyl, aryloxy, or combinations thereof.

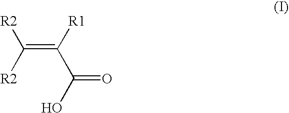

Some examples of α,β-unsaturated carboxylic acids may include acrylic acid, crotonic acid, isocrotonic acid, methacrylic acid, sorbic acid, cinnamic acid, maleic acid, fumaric acid, or combinations thereof. In some embodiments, the α,β-unsaturated carboxylic acid is acrylic acid or methacrylic acid.

A second inlet stream may comprise a halogenating agent. The halogenating agent includes compounds having at least one halogen atom. The halogenating agent may be a liquid, a gas, or a solid. For example, the halogenating agent can react with an α,β-unsaturated carboxylic acid in a chemical reaction. Halogenating agents are further described in Larock, R. C., Comprehensive Organic Transformations: A Guide to Functional Group Preparations, VCH Publishers, Inc., (1989). In some instances, specific halogenating agents may be more selective for converting the α,β-unsaturated carboxylic acid to an α,β-unsaturated carbonyl halide than other halogenating agents. Some examples of halogenating agents include organic chlorides, inorganic chlorides, inorganic bromides, acyl chlorides, or combinations thereof. More specifically, some halogenating agents can include oxalyl chloride, oxalyl bromide, thionyl chloride, phosgene, triphosgene, phosphorous trichloride, phosphorous tribromide, phosphorous pentachloride, phosphorous pentabromide, carbon tetrachloride/triphenylphosphine, or combinations thereof. In some embodiments, the halogenating agent can be oxalyl chloride or thionyl chloride.

In one embodiment, oxalyl chloride may be more selective than thionyl chloride in converting the acid functional group of an α,β-unsaturated carboxylic acid to an acid chloride functional group under similar processing conditions. That is, an increase in the percent yield of the α,β-unsaturated carbonyl halide, and a decrease in the percent of a Michael addition product and the acid addition product may be observed with oxalyl chloride. For example, compared to other halogenating agents, the use of oxalyl chloride can result in at least a 10 percent increase in the percent yield of the α,β-unsaturated carbonyl halide with less than 1 mole percent of the Michael addition product and less than 1 mole percent of the acid addition product formed.

The first inlet stream or an optional third stream may comprise a catalyst as a reactant for forming a α,β-unsaturated carbonyl halide. The catalyst may be delivered in the first inlet stream with an α,β-unsaturated carboxylic acid, such that the catalyst is dissolved or dispersed in the α,β-unsaturated carboxylic acid. The catalyst may be added as a liquid, where the catalyst is free of solvent. In some embodiments, the catalyst may be dispersed in a solvent, or dissolved in a solvent. The presence of a catalyst can increase the kinetics of a chemical reaction without increasing the reaction temperatures. In one embodiment, the presence of a catalyst may improve the percent yield of the α,β-unsaturated carbonyl halide relative to a similar halogenation reaction without the catalyst.

Some examples of catalysts include amides, imides, imidazoles, triazoles, pyridines, oximes, isocyanates, thiazoles, pyrazoles, or combinations thereof. More specifically, some catalysts may include dimethylformamide (DMF), acetamide, methyl imidazole, imidizole, pyridine, or combinations thereof. In one embodiment, the catalyst may be dimethylformamide.

The α,β-unsaturated carboxylic acid and the halogenating agent may flow into the reaction chamber such that the moles of the α,β-unsaturated carboxylic acid exceeds the moles of the halogenating agent. A percent excess of the moles of the α,β-unsaturated carboxylic acid relative to the moles of the halogenating agent may be greater than an equimolar 1:1 stoichiometry. The percent excess of the moles of the α,β-unsaturated carboxylic acid relative to the moles of the halogenating agent may be at least 0.01, at least 0.05, at least 0.1, at least 1, or at least 1.5 percent. The percent excess of the moles of the α,β-unsaturated carboxylic acid relative to the moles of the halogenating agent may be up to 10 percent, up to 8 percent, up to 6 percent, or up to 5 percent. The percent excess of the moles of the α,β-unsaturated carboxylic acid relative to the moles of the halogenating agent may be in a range of 0.01 to 10, 0.05 to 8, 0.1 to 6, or in a range of 0.1 to 5 percent. In some embodiments, the percent excess of moles of the α,β-unsaturated carboxylic acid is greater than the moles of the halogenating agent.

The halogenating agent and the α,β-unsaturated carboxylic acid may flow into the reaction chamber such that the moles of the halogenating agent exceeds the moles of the α,β-unsaturated carboxylic acid. A percent excess of the moles of the halogenating agent relative to the moles of the α,β-unsaturated carboxylic acid may be greater than an equimolar 1:1 stoichiometry. The percent excess of the moles of the halogenating agent relative to the moles of the α,β-unsaturated carboxylic acid may be at least 0.01 percent, at least 0.1 percent, at least 1 percent, at least 2 percent, or at least 3 percent. The percent excess of the moles of the halogenating agent relative to the moles of the α,β-unsaturated carboxylic acid may be up to 10 percent, up to 8 percent, up to 6 percent, or up to 5 percent. The percent excess of the moles of the halogenating agent relative to the moles of the α,β-unsaturated carboxylic acid may be in a range of 0.01 to 10, 0.1 to 8, 1 to 6, or in a range of 2 to 5 percent. In some embodiments, the percent excess of moles of the halogenating agent is greater than the moles of the α,β-unsaturated carboxylic acid.

A catalyst may flow as a first inlet stream with an α,β-unsaturated carboxylic acid into a reaction chamber. The mole percent of catalyst relative to the number of moles of the α,β-unsaturated carboxylic acid may be at least 0.01 mole percent, 0.1 mole percent, 1 mole percent, or at least 2 mole percent. The mole percent of catalyst relative to the number of moles of the α,β-unsaturated carboxylic acid may be up to 5 mole percent, up to 4 mole percent, or up to 3 mole percent. The mole percent of catalyst relative to the number of moles of the α,β-unsaturated carboxylic acid may be in a range of 0.01 to 5 mole percent, 0.1 to 4 mole percent, or in a range 0.5 to 3 mole percent.

Referring to Reaction A, an α,β-unsaturated carboxylic acid, a halogenating agent, and a catalyst mix with one another in a flow-through microreactor to form an α,β-unsaturated carbonyl halide. As the reaction proceeds, a gas may evolve as the α,β-unsaturated carboxylic acid is converted to the α,β-unsaturated carbonyl halide, and the halogenating agent is consumed. In one embodiment, a gas may be added as a reactant to an inlet stream.

Halogenation (e.g., with a halogenating agent) of an α,β-unsaturated carboxylic acid with a catalyst is usually exothermic. In some embodiments the α,β-unsaturated carboxylic acid, the halogenating agent, and the catalyst are reactants of a solventless reaction. Efficient dissipation of heat in a flow-through microreactor may contribute to forming reaction products at a lower reaction temperature, and the use of catalysts may contribute to achieving increased reaction rates. The result of lower reaction temperatures may reduce the amount of byproducts, and increase the percent yield of the reaction product. In one embodiment, the α,β-unsaturated carbonyl halide may be formed having a percent yield of at least 75 percent, at least 85 percent, or at least 90 percent. An α,β-unsaturated carbonyl halide may be formed having a percent yield up to 99.9 percent, up to 96 percent, or up to 95 percent. The percent yield of the α,β-unsaturated carbonyl halide may be in a range of 75 to 99.9 percent, 85 to 99.9 percent, or 90 to 99 percent. The percent yield of the α,β-unsaturated carbonyl halide can be determined based on the compounds in a reaction stream without purification. The reaction stream may comprise a reaction compound, unreacted reactants, Michael addition products, acid addition products and other byproducts. In some embodiments, a byproduct, such as a Michael addition product, may be less than 10 mole percent.

In some embodiments, a Michael addition product may be the dominant byproduct in a reaction stream where a catalyst was a reactant in an inlet stream. In other embodiments, the Michael addition product may be one of many byproducts present in a reaction stream where a catalyst was not in an inlet stream.

In one embodiment, an acrylic acid reacted with an oxalyl chloride in the presence of dimethyl formamide in a solventless reaction formed an acryloyl chloride having a percent yield of at least 96 percent without purification of the reaction stream. The Michael addition product or the acid addition product was less than 3 mole percent.

In another embodiment, a methacrylic acid reacted with an oxalyl chloride and dimethyl formamide in a solventless reaction formed a methacryloyl chloride having a percent yield of greater than 99 percent without purification of the reaction stream. The Michael addition product or the acid addition product was not detectable by ¹H NMR.

In another aspect, a process is provided for forming an α,β-unsaturated carbonyl halide. The reactants for the process comprise an α,β-unsaturated carboxylic acid provided in a first inlet stream, a halogenating agent comprising oxalyl chloride in a second inlet stream, and a catalyst in the first inlet stream or in an optional third inlet stream. The first inlet stream, the second inlet stream, and the optional third inlet stream are flowed into a reaction chamber of a flow-through microreactor. The process further includes forming a reaction product comprising an α,β-unsaturated carbonyl halide having a percent yield greater than 90 percent. The reaction can be conducted in the presence or absence of a solvent.

In one embodiment, at least one of the first inlet stream, the second inlet stream, or the optional third inlet stream comprises a solvent. For example, the solvent may be added to the reactants to dilute, dissolve, or disperse reactants. The solvent may flow with the reactants into the reaction chamber. Some examples of solvents can include ethers, esters, aromatics, alkanes, ketones, heterocyclics, heteroaromatic cyclics, chlorinated alkanes, or combinations thereof. Suitable solvents, for example, may include tetrahydrofuran, methylene dichloride, ethylene dichloride, o-dichlorobenzene, propylene carbonate, chloroform, trichloroethylene, 1,4-dioxane, ethyl acetate, chlorobenzene, toluene, benzene, diethyl ether, 2-methyl hydrofuran, carbon tetrachloride, hexane, and combinations thereof.

In another embodiment, a solventless process is provided for forming an α,β-unsaturated carbonyl halide. The process further includes forming a reaction product comprising an α,β-unsaturated carbonyl halide having a percent yield greater than 90 percent, wherein the halogenating agent is oxalyl chloride.

Microreactors can be used for synthesizing specialty and fine chemicals. The small characteristic lengths of these microreactors, typically less than several millimeters, can be used for reactions that are highly exothermic, have hazardous reagents or byproducts, occur at high or low temperatures, and/or for reactions with unstable intermediates.

Microreactors having structures (e.g., microstructures) with small dimensions (e.g., inlets, inlet regions, inlet channels, reaction chambers, residence time channels, and outlets) may have capabilities that can exceed conventional macroscopic systems or large scale reactor systems. Some examples of microreactors that are commercially available include microreactors from Ehrfeld Mikrotechnik GmbH, Germany (model # 0211-02-0311-F); Alfa Laval, Sweden (plate reactor); and CPC-Cellular Process Chemistry Systems, Germany (CYCTOS Lab Systems). Some of the capabilities of microreactors may manifest themselves in the enhancement of the physical transport phenomena underlying many chemical processes, and the ability to control and tune them. The inherently small length scales (and high surface-to-volume ratios) involved expedite heat and mass transfer to a large degree such that aggressive processing conditions not feasible on a macroscopic scale may be possible in microreactors.

Flow-through processing based on fluid chemistry is one advantage for using flow-through microreactors. Small channel size and high surface to volume ratios make these devices more efficient than large scale batch reactors in heat and mass transfer. Improved heat transfer allows reactions to be run at room temperature, rather than under cryogenic conditions. Rapid and effective mixing brings reactants into contact for better conversion. Faster reactions, along with controlled residence times and temperatures, can generate desired reaction products without or substantially without unwanted byproducts, thus resulting in higher selectivity and yields.

Microreactors of this disclosure are described in terms of their relative dimensions, and in some instances, described in terms of volume or capacity. The volume of a microreactor may be described as interconnected microstructures having a capacity from an inlet for the addition of reactants extending to an outlet for a reaction stream. The dimensions of the microreactors and corresponding microstructures may be described as a range (e.g., millimeters or sub-millimeters), such that the range may be altered to accommodate other microreactor designs. Similarly, the volume of microreactors and its corresponding microstructures may be altered as dictated by design and application needs.

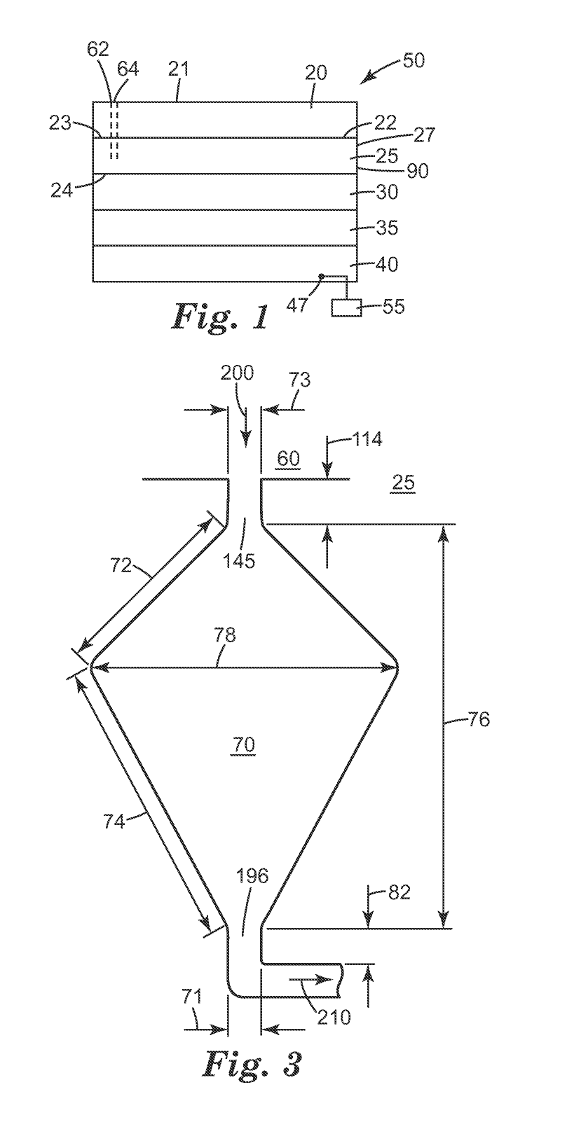

FIG. 1 illustrates a cross-section of an exemplary flow-through microreactor 50 having a layered assembly. Flow-through microreactor 50 comprises a first layer 20, a second layer 25, a third layer 30, a fourth layer 35, and an optional resistive heating element 40. The first layer 20, the second layer 25, the third layer 30, the fourth layer 35, and the optional resistive heating element 40 may have holes (not shown) for inserting fasteners (not shown). The first layer 20 seals the microstructures (not shown) of the second layer 25 of the microreactor 50. The second layer 25 comprises microstructures (e.g., inlets, inlet channels, reaction chambers, residence time channels, outlets, and combinations thereof) of a flow-through microreactor 50. The second layer 25 is capable of being milled or microfabricated. The third layer 30 provides support for the second layer 25 and the first layer 20. The third layer 30 also seals the microstructures of the second layer 25, and provides efficient heat transfer for the second layer 25 and the fourth layer 35. The fourth layer 35 provides support for the first layer 20, the second layer 25, and the third layer 30. The fourth layer 35 may be constructed to provide for efficient heat transfer from the resistive heating element 40 through the third layer 30 to the second layer 25 of the layered assembly. The optional resistive heating element 40 has an attached thermocouple 47, such that the attached thermocouple 47 is in communication with an external temperature controller 55 allowing for digital temperature control of the flow-through microreactor 50.

Chemical compatibility, temperature stability, and ease of fabrication are some of the considerations for selecting materials for constructing a microreactor. Some materials available for use in microreactors may include silicon, quartz, glass, metals, polymeric materials, and combinations thereof. A range of channel microfabrication methods such as photolithography, hot embossing, powder blasting, injection molding, laser machining, or micromachining may be used. Channel microfabrication of a second layer 25, for example, of a flow-through microreactor 50 may be used to develop microstructures. Layers adjacent to the second layer 25, such as a first layer 20 and a third layer 30, may comprise similar or different materials for a flow-through microreactor 50. The selection of material may be dependent on the application and user requirements. Some examples of microstructures include inlets, inlet channels, reaction chambers, pre-residence time channels, residence time channels, outlets, miscellaneous microstructures, and combinations thereof. Some examples of miscellaneous microstructures may include ports for mixing means, sample access, and analytical equipment interfacing.

Referring to FIG. 1, the first layer 20 has a first surface 21 and a second surface 22. The second layer 25 has a third surface 23 and a fourth surface 24. The second surface 22 is adjacent to the third surface 23 of the flow-through microreactor 50. First inlet 62 and second inlet 64 may have a cylindrical dimension forming openings extending through the first surface 21 and second surface 22 of the first layer 20. In some embodiments, the first inlet 62 and the second inlet 64 each independently may extend through at least one side of a second layer 25. The first inlet 62 and the second inlet 64 may both extend into at least a portion of the second layer 25 through the third surface 23. The second layer 25 may contain microstructures (not shown) of the flow-through microreactor 50. The first side 27 of second layer 25 comprises an outlet 90. The optional resistive heating element 40 can have an attached thermocouple 47 interfaced with an external temperature controller 55. Microreactor 50 may optionally be interfaced with one or more microreactors for upstream or downstream processing.

The first layer 20 of microreactor 50 comprises a first surface 21 and a second surface 22. The first layer 20 is adjacent to the third surface 23 of the second layer 25. The first surface 21 has openings (not shown) for the first inlet 62 and the second inlet 64. The first inlet 62 and the second inlet 64 extend from the first surface 21 to the second surface 22. The first inlet 62 and the second inlet 64 further extend through third surface 23, and into a portion of the second layer 25. The diameter of the openings (not shown) of the first inlet 62 and the second inlet 64 may be of the same dimensions or different dimensions. The diameter of the openings (not shown) of the first inlet 62 and the second inlet 64 may be sufficient to allow for tubing, for example, polytetrafluoroethylene (PTFE), to be interfaced with the flow-through microreactor 50. The tubing (not shown) can provide for the flow of reactants into the flow-through microreactor 50. Any suitable thickness can be used for the first layer 20. This thickness is often in a range of 0.01 to 1 cm, 0.02 to 0.5 cm, or in a range of 0.03 to 0.1 cm. In one embodiment, the thickness of the first layer 20 is approximately 0.047 cm. The first layer 20 is constructed of materials that are passive to gases and corrosive materials, and that have high temperature stability. Some examples of materials which have these properties include nickel, copper, aluminum, alloys (e.g., stainless steel, HASTELLOY (a trade designation of Haynes International), MONEL (a trade designation of Special Metals Corporation)), or combinations thereof. In one embodiment, the first layer 20 comprises stainless steel.

A second layer 25 comprises a third surface 23 and a fourth surface 24. The second layer 25 is adjacent to the first layer 20 and the third layer 30. The second surface 22 of the first layer 20 is adjacent to the third surface 23 of the second layer 25, and the third layer 30 is adjacent to the fourth surface 24. First inlet 62 and second inlet 64 both extend through the third surface 23 and into the microstructures (not shown) of the flow-through microreactor 50. An outlet 90 is located on the first side 27 of the second layer 25. The first inlet 62 and the second inlet 64 independently may have openings in any suitable range size. In some embodiments, these openings are in a range of 1 mm to 3 mm. In one embodiment, the openings of both the first inlet 62 and the second inlet 64 are approximately 2 mm.

The second layer 25 comprises microstructures (not shown), such that the microstructures are adjacent to one another and interconnect to form continuous regions which allow for the flow of reactants and a reaction stream. The second layer 25 comprises a material capable of being milled or microfabricated, such that the material has high temperature stability, excellent chemical resistance and passivity to gases. Some examples of materials for the second layer 25 may include polytetrafluoroethylene (PTFE), polychlorotrifluouroethylene (PCTFE), perfluoroalkoxy polymer (PFA), polyvinylidene difluoride (PVDF), or combinations thereof. In one embodiments, the second layer 25 is polytetrafluoroethylene (PTFE). Any suitable thickness for the second layer 25 can be used. In some embodiments, the thickness of the second layer 25 can be at least 0.1 cm, at least 0.25 cm, at least 0.50 cm, or at least 0.65 cm. The thickness of the second layer 25 can be up to 1.5 cm, up to 1.25 cm, or up to 1.20 cm. The thickness of the second layer 25 can be in a range of 0.1 cm to 1.5 cm, 0.25 cm to 1.25 cm, or in a range of 0.65 to 1.20 cm. In one embodiment, the thickness of the second layer 25 is approximately 1.17 cm.

A third layer 30 is located adjacent to the fourth surface of the second layer 25. A fourth layer 35 is located on an opposite surface of the third layer 30. The third layer 30 also has chemical resistivity to the reactants that may come into contact with the third layer 30. The third layer 30 can be constructed of materials which are passive to gases, resistance to corrosive materials, and have high temperature stability. Some examples of materials having these properties include nickel, copper, aluminum, alloys (e.g., stainless steel, HASTELLOY or MONEL), or combinations thereof. In some embodiments, the third layer 30 comprises MONEL. Any suitable thickness can be used for the third layer 30. In some embodiments, the thickness of the third layer 30 can be in a range of 0.01 to 1 cm, 0.02 to 0.5 cm, or in a range of 0.03 to 0.1 cm. In one embodiment, the thickness of the third layer 30 is approximately 0.047 cm.

A fourth layer 35 is located between the third layer 30 and the optional resistive heating element 40 of the flow-through microreactor 50. The fourth layer 30 can be constructed of materials which are passive to gases, resistant to corrosive materials, and have high temperature stability. Some materials having these properties include nickel, copper, aluminum, alloys (e.g., stainless steel, HASTELLOY or MONEL), or combinations thereof. In some embodiments, the fourth layer 30 comprises aluminum or copper. Optionally, the fourth layer 35 may be fabricated for the attachment or insertion of heating elements to form a resistive heating element. Any suitable thickness can be used for fourth layer 35. In some embodiments, the thickness of the fourth layer 35 may be at least 0.01 cm, at least 0.02 cm, or at least 0.03 cm. The thickness of the fourth layer 35 may be up to 1 cm, up to 0.5 cm, or up to 0.1 cm. The thickness of the fourth layer 35 may be in a range of 0.01 to 1 cm, 0.02 to 0.5 cm, or in a range of 0.03 to 0.1 cm. In one embodiment, the thickness of the fourth layer 35 is approximately 0.047 cm.

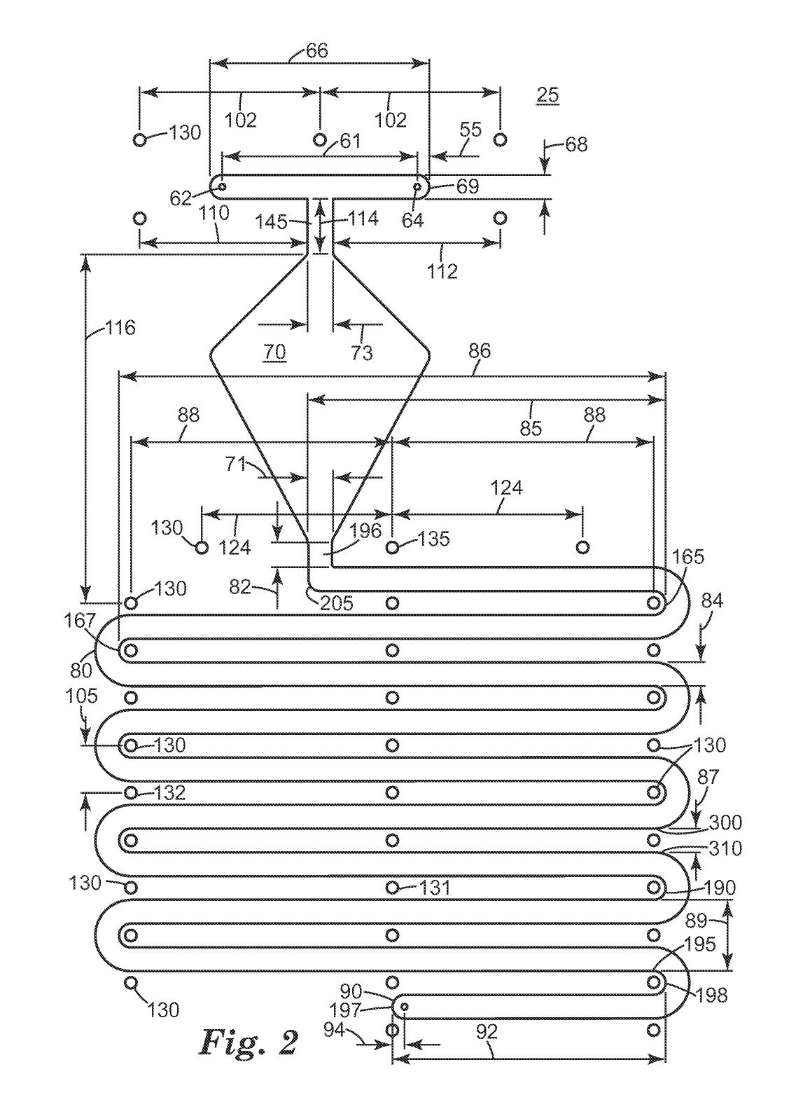

The layered assembly of a flow-through microreactor 50 of FIG. 1 is assembled to prevent the loss of reactants or a portion of the reaction stream, to prevent the introduction of moisture, and to seal the flow-through microreactor under pressure. The flow-through microreactor 50 may comprise drilled holes 130 for attaching the multiple layers adjacent to one another. FIG. 2 illustrates the location of the drilled holes 130 in a second layer 25. A number of drilled holes 130 are located in the second layer 25 The drilled holes 130 may extend through a first layer 20, through a second layer 25, through a third layer 30, through a fourth layer 35, and optionally through a resistive heating element 40 attached to the fourth layer 35 of FIG. 1. The optional resistive heating element 40 may not be attached to the microreactor 50 with fasteners. Any suitable diameter can be used for the drilled holes 130. In some embodiments, the diameter of the drilled holes 130 can be in a range 1 mm to 5 mm. In one embodiment, the diameter of the drilled holes 130 may be in a range of 3.75 mm to 4 mm. In another embodiment, the drilled holes 130 through the first layer 20, the second layer 25, the third layer 30, and extending through the fourth layer 35 can be threaded.

An exemplary flow-through microreactor 50 comprising a second layer 25 is illustrated in FIG. 2. The second layer 25 contains microstructures which may be formed as described above. A third surface 23 of the second layer 25 (illustrated in FIG. 1) may be milled or microfabricated to produce microstructures. A fourth surface 24 of the second layer 25 may be optionally milled or microfabricated to produce similar microstructures. The microstructures (e.g. first inlet 62, second inlet 64, inlet region 60, inlet channel 145, reaction chamber 70, pre-residence time channel 196, residence time channel 80, and outlet 90) of the second layer 25 interconnect to form a continuous channel for the flow of reactants. The continuous channel provides for flow of the reactants from an entry location to the recovery of a reaction product in a reaction stream at an exit location. A first inlet 62 and a second inlet 64 contact an inlet region 60 (illustrated in FIG. 3). The inlet region 60 contacts an inlet channel 145 which extends to a reaction chamber 70. The reaction chamber 70 provides flow of the reactants as a reaction stream to a residence time channel 80, having an outlet 90 for the exiting reaction stream. The height (e.g., depth) of the microstructures of FIG. 2 within the second layer 25 may have varying dimensions. The height of the microstructures may not be limited to only one microstructure, but may be applicable to more than one of the microstructures within the second layer 25. Any suitable height can be used for the inlet channel 145 and the residence time channel 80, for example. In some embodiments, the height (e.g., depth) of the inlet channel 145 and the residence time channel 80 independently may be at least 0.01 cm, at least 0.025 cm, or at least 0.04 cm. The height of the inlet channel 145 and the residence time channel 80 independently may be up to 0.25 cm, up to 0.15 cm, or up to 0.1 cm. The height of the inlet channel 145 and the residence time channel 80 independently may be in a range of 0.01 cm to 0.25 cm, 0.025 to 0.15 cm, or up to 0.025 to 0.1 cm. Any suitable width can be used for the inlet channel 145 and the residence time channel 80, for example. The width of the microstructures may not be limited to only one microstructure, but may be applicable to more than one of the microstructures within the second layer 25. In some embodiments, the width of the inlet channel 145 and the residence time channel 80 independently may be at least 0.01 cm, at least 0.05 cm, or at least 0.075 cm. The width of the inlet channel 145 and the residence time channel 80 independently may be up to 3 cm, up 2 cm, or up to 1.75 cm. The width of the inlet channel 145 and the residence time channel 80 independently may be in a range of 0.01 cm to 3 cm, 0.05 to 2 cm, or in a range of 0.075 to 1.75 cm. In one embodiment, the height of the inlet channel 145 and the residence time channel 80 is approximately 0.0585 cm, and the width of the inlet channel 145 and the residence time channel 80 is approximately 0.161 cm. The length of the channels (e.g., the inlet channel 145 and the residence time channel 80) may be in a range of 2 mm to 250 cm.

Materials such as liquids, gases, dispersions, and combinations thereof may flow as inlet streams. Materials such as reactants may be introduced through inlets, where the inlets provide access for delivery of the inlet streams to a flow-through microreactor 50. The inlet streams comprising the materials may flow into interconnected microstructures to deliver the materials at a predetermined flow rate into a microreactor. The inlet streams may flow under anhydrous conditions, such that the flow-through microreactor 50 may be sealed to prevent the infiltration of water or other undesirable materials. The inlet streams can be flushed with an inert gas (e.g., argon, nitrogen, helium, or combinations thereof) prior to addition and during the addition of reactants to preclude side reactions yielding byproducts. In one embodiment, the inlet streams comprise reactants which flow into a reaction chamber to form a solventless reaction. The flow-through microreactor 50 can have sufficient volume to accommodate the reactants for a chemical reaction, a coating or material modification. The volume can be described as the capacity of the microstructures for containing flowable materials from an inlet (e.g. the first inlet 62 or the second inlet 64) to an outlet 90 of the flow-through microreactor 50. Any suitable volume for the flow-through microreactor 50 can be used. In some embodiments, the volume of the flow-through microreactor 50 may be of at least 0.01 ml, of at least 0.1 ml, of at least 1 ml, of at least 3 ml, or of at least 5 ml. The volume of the flow-through microreactor 50 may be up to 100 ml, up to 75 ml, up to 50 ml, up to 35 ml, or up to 25 ml. The volume of the flow-through microreactor 50 may be in a range of 0.01 to 100 ml, 0.01 to 75 ml, 0.01 to 50, or in a range of 0.01 to 20 ml.

A flow-through microreactor 50 may have a plurality of inlets (e.g. first inlet channel 62) for delivering materials or reactants as inlet streams to a microstructure (e.g., inlet region, or an inlet channel). Any suitable number of inlets can be used to deliver materials or reactants to a microstructure of a flow-through microreactor 50. In some embodiments, the number of inlets may be at least two, at least three, or at least 5 inlets for delivering reactants to the flow-through microreactor 50. The flow-through microreactor 50 may have up to 10, up to 8, or up to 7 inlets. The flow-through microreactor 50 may have a number of inlets, such that the number may be in a range of 2 to 10 inlets, 2 to 8 inlets, or 2 to 7 inlets. The microstructures of the flow-through microreactor 50 may have different geometrical configurations, and varying dimensions to contain reactants or materials.

The configurations of microstructures of the flow-through microreactor 50 may vary. Any suitable configuration can be used for a microstructure. In some embodiments, an inlet channel 145 can have configurations including geometries such as linear, spherical, ellipsoidal, serpentine, and combinations thereof. In some embodiments, the configuration of the inlet channel 145 may be linear or serpentine. An inlet channel 145 provides a path for the inlet streams to flow from an inlet region 60 to a reaction chamber 70. The inlet channels have sufficient height and width for flowing reactants to a reaction chamber 70 for mixing to form the reaction stream, which can flow into a residence time channel 80.

The inlet region 60 (illustrated in FIG. 3) comprises a first inlet 62 and a second inlet 64 as illustrated in FIG. 2. The first inlet stream may be delivered through the first inlet 62, a second inlet stream may be delivered through a second inlet 64, and an optional third stream may be delivered through an optional third inlet (not shown). The inlet streams flow into the inlet region 60, and then flow into an inlet channel 145. The inlet streams may be added to the first inlet 62 and the second inlet 64 under pressure (e.g., pump). The inlet streams may be heated or cooled with a resistive heating element 40 attached to a thermocouple 47 (illustrated in FIG. 1) to control the temperature of the reactants of the first inlet stream, the second inlet stream, and an optional third stream prior to entering into a reaction chamber 70 of a flow-through microreactor 50. Any suitable temperature can be used for the inlet streams. In some embodiments, the temperature of the inlet streams may be at least −20° C., at least 0° C., at least 15° C., or at least 20° C. The temperature of the inlet streams may be up to 100° C., up to 75° C., up to 50° C., or up to 25° C. The temperature of the inlet streams may be in a range of −20° C. to 100° C., 0° C. to 75° C., or in a range of 15° C. to 50° C. In one embodiment, the temperature of the inlet streams is 25° C.

An inlet stream may comprise one or more reactants. The reactants that are in the same inlet stream are typically selected so that they do not react with each other. The reactants that may react with one another may be kept in separate inlet streams, for example, in a first inlet stream and in a second inlet stream. The first inlet stream can be delivered to a first inlet 62, and a second inlet stream can be delivered to a second inlet 64. The reactants of the first inlet stream may react with the reactants of the second inlet stream in the reaction chamber 70. The construction of the inlets, inlet regions, reaction chamber, and the residence time channels of the flow-through microreactor 50 may be designed and used to prolong or to provide sufficient residence time for chemical reactions to occur in order to achieve high percent yields (e.g., high percent conversion).

FIG. 2 illustrates some exemplary microstructures microfabricated within a second layer 25 of a flow-through microreactor 50 (illustrated in FIG. 1). A first inlet 62 and a second inlet 64 are located within inlet region 60 (illustrated in FIG. 3), such that the first inlet 62 and the second inlet 64 are separated by a first distance 61. The first inlet 62 and the second inlet 64 may have a diameter of 1 to 3 mm. The first distance 61 may be in a range of 20 to 35 mm. The inlet region 60 may have a second distance 66 and a third distance 68. The second distance 66 may be in a range of 30 to 45 mm, and the third distance 68 may be in a range of 0.3 to 1.5 mm. A fourth distance 69 of the inlet region 60 resides between the second inlet 64 and the inlet region wall 55. The fourth distance 69 may be in a range of 2 to 5 mm. A fifth distance 102 between drilled holes 130 outside of the inlet region 60 may be in a range of 10 to 20 mm. The inlet region 60 can be connected to an inlet channel 145.

The inlet channel 145 may have a sixth distance 114 extending from the inlet region 60 to a reaction chamber 70. A seventh distance 73 describes the width of the inlet channel 145. The sixth distance 114 may be in a range of 1 to 3 mm, and the seventh distance 73 may be in a range of 5 to 15 mm. An eighth distance 110 and a ninth distance 112 each describe the distance between drilled holes 130 outside of the inlet channel 145. The eighth distance 110 and the ninth distance 112 may each be in a range of 20 to 40 mm. The inlet channel 145 may be connected to a reaction chamber 70. A twenty-second distance 116 describes the distance from an intersection of the inlet channel 145 and the reaction chamber 70 extending the length of the reaction chamber 70, parallel to the reaction stream flow, through the pre-residence time channel 196 to a drilled hole 130 adjacent to the residence time channel 80.

A tenth distance 82 of a pre-residence time channel 196 connects the reaction chamber 70 to a residence time channel 80. The tenth distance 82 may be in a range of 1 to 4 mm. An eleventh distance 71 describes the width of the pre-residence time channel 196. The eleventh distance 71 may be in a range of 5 to 15 mm. The pre-residence time channel 196 may be further connected to residence time channel 80. A twenty-first distance 124 may be the distance between drilled hole 130 and a fifth drilled hole 135. The twenty-first distance 124 may be in a range of 25 to 35 mm.

The residence time channel 80 may have a serpentine configuration extending to an outlet 90. The residence time channel 80 may have a twelfth distance 84, a thirteenth distance 85, and a fourteenth distance 86. The twelfth distance 84 may be the width of the residence time channel 80. The twelfth distance 84 may be in a range of 5 to 15 mm. The thirteenth distance 85 may be a distance from the pre-residence time channel bend 205 to a first bend 165. The thirteenth distance 85 may be in a range of 40 to 50 mm. The fourteenth distance 86 may be a distance from the first bend 165 to a second bend 167. The fourteenth distance 86 may be in a range from 80 to 100 mm. A fifteenth distance 88 may extend from a drilled hole 130 to a second drilled hole 131. The fifteenth distance 88 may be in a range of 25 to 35 mm. A sixteenth distance 89 may extend from a first interior edge 190 to a second interior edge 195 of the residence time channel 80. The sixteenth distance 89 may be in a range of 20 to 30 mm. A seventeenth distance 87 may extend from a first exterior edge 300 to a second exterior edge 310 of the residence time channel 80. The seventeenth distance 87 may be in a range of 5 to 15 mm. An eighteenth distance 105 may extend from a drilled hole 130 to a second drilled hole 132 adjacent to the residence time channel 80. The eighteenth distance 105 may be in a range of 15 to 25 mm. The residence time channel 80 may extend to an outlet 90.

The outlet 90 may be located at a nineteenth distance 94 from a third interior edge 197 of the residence time channel 80. The nineteenth distance 94 may be in a range of 1 to 5 mm. A twentieth distance 92 may extend from a fourth interior edge 198 to the third interior edge 197. The twentieth distance 92 may be a range of 35 to 45 mm. The outlet 90 may have a diameter in a range of 1 to 3 mm.

Inlet streams comprising reactants may enter a first inlet 62 and a second inlet 64 to be delivered to an inlet region 60 of a flow-through microreactor 50 via pumps, more particularly syringe pumps, piston pumps, gear pumps, a pressure pot with a mass flow controller, or combinations thereof. Fluid flow in the flow-through microreactor 50 may be pressure driven and pulse free for precise delivery of exact stoichiometric amounts of reagents with the desired residence times to meet production requirements. HPLC and or syringe pumps can be used to achieve this flow characteristic. The inlet streams may be metered into the flow-through microreactor 50 at the first inlet 62 and the second inlet 64 each independently having any suitable flow rate. In some embodiments, the flow rate is at least 0.010 ml/minute, at least 0.05 ml/minute, or 0.1 ml/minute. The inlet streams can be metered into the flow-through microreactor 50 at the first inlet 62 and the second inlet 64 each independently having a flow rate up to 10 ml/minute, up to 5 ml/minute, or up to 1 ml/minute. The inlet streams can be metered into the flow-through microreactor 50 at the first inlet 62 and the second inlet 64 each independently having a flow rate, for example, in a range of 0.01 ml/minute to 10 ml/minute, 0.05 ml/minute to 5 ml/minute, or in a range of 0.1 ml/minute to 1 ml/minute. The microstructures of the second layer 25 have specified dimensions which typically allow for accurate control over very low flow rates. Fine control over the flow rate with precise control over the residence time can provide a highly controllable flow-through microreactor.

FIG. 3 illustrates an exemplary reaction chamber 70 having a volume sufficient for retaining the inlet streams having a first flow 200. The reaction chamber 70 can have a high surface area to volume ratio to allow for efficient heat dissipation to the walls of a flow-through microreactor 50 in the event of exothermic reactions, reducing the tendency for side products to form. The high surface area to volume ratio may allow for efficient transfer of heat into the reacting medium from external sources, such as may be required in endothermic reactions, or in reaction initiation. The flow-through microreactor 50 can further provide an efficient means for heat sinking from or heat sourcing to the reaction chamber. The high surface area to volume ratio further provides for a high interfacial area for chemical transfer compared with the volume of the fluid to be reacted. Further, it may be possible to use substantially reduced amounts of heat dissipating solvents or no solvents compared with the amounts used in conventional methods.

Inlet streams comprising reactants of a first flow 200 may be delivered into a reaction chamber 70 as illustrated in FIG. 3. Upon contact with each other, the reactants may react with one another to form a reaction product. In some embodiments, the reactants are provided for a solventless reaction. The temperature of the reactants in the reaction chamber 70 may be at least −20° C., at least 0° C., or at least 20° C. The temperature of the reactants in the reaction chamber 70 may be up to 100° C., up to 80° C., or up to 60° C. The temperature of the reactants in the reaction chamber 70 may be in a range of −20° C. to 100° C., 0° C. to 80° C., or in a range of 20° C. to 60° C. The temperature of the reaction chamber 70 may be controlled with a heating or a cooling means. The temperature of the reactants within the reaction chamber 70 may increase due to an exotherm generated from the reaction of one reactant with another reactant. The reaction product may be formed over a period of time as the reactants flow and are mixed in the reaction chamber 70. The reaction product may continue to be formed as the reaction stream flows from the reaction chamber 70 into a pre-residence time channel 196 as a second flow 210. The temperature of the reaction chamber 70 may be sufficient for the reactants to react in a chemical reaction for forming a reaction product.

Dimensions of a reaction chamber 70 can be provided to enable sufficient mixing of the reactants for forming a reaction product in a reaction stream. The reaction chamber 70 may have geometrical configurations such as spherical, circular, ellipsoidal, diamond, linear, and combinations thereof. In one embodiment, the geometrical configuration of the reaction chamber 70 is a diamond.

The volume of a reaction chamber 70 may be one effective to ensure mixing of the reactants to form a reaction product. The volume or capacity of the reaction chamber 70 providing for efficient mixing of the inlet streams may be characterized by an aspect ratio. The aspect ratio of a reaction chamber 70 may be defined as the width of the reaction chamber 70 relative to the height of the reaction chamber 70 (e.g., width:height). For example, a first aspect ratio can be defined as the ratio of a first chamber width 78 as illustrated in FIG. 3 to the height of the reaction chamber 70 (e.g., 78:height). The first chamber width 78 can be described as the width of the reaction chamber 70 orthogonal to a first flow 200. A second aspect ratio can be defined as the ratio of a second chamber width 76 to the height of the reaction chamber 70 (e.g., 76:height). The second chamber width 76 can be described as the width of the reaction chamber 70 parallel to a first flow 200. The height of the reaction chamber 70 may be described by the depth of the microstructure within the second layer 25. Any suitable height can be used for the height of the reaction chamber 70. In some embodiments, the height of the reaction chamber 70 may be at least 0.01 cm, at least 0.025 cm, or at least 0.04 cm. The height of the reaction chamber 70 may be up to 0.25 cm, up to 0.15 cm, or up to 0.1 cm. The height of the reaction chamber 70 may be in a range of 0.01 cm to 0.25 cm, 0.025 to 0.15 cm, or up to 0.025 to 0.1 cm.

An exemplary reaction chamber 70 of the second layer 25 comprises a diamond shaped geometry in FIG. 3. A first inlet stream, a second inlet stream, and an optional third stream having a first flow 200 may be delivered from an inlet region 60 to an inlet channel 145, and flowed into the reaction chamber 70. The inlet channel 145 may have a width of a seventh distance 73 and a length of a sixth distance 114. The reaction chamber 70 may have a first chamber side 72, and a second chamber side 74. The first chamber side 72 may have a length in a range of 10 to 15 mm, and the second chamber side 74 may have a length in a range of 15 to 25 mm. The reaction chamber 70 may have a first chamber width 78 orthogonal to the first flow 200. Similarly, reaction chamber 70 may have a second chamber width 76 parallel to the first flow 200. The reaction chamber 70 may have a first chamber width 78 in a range of 40 to 60 mm. A second chamber width 76 may be in a range of 60 to 120 mm. A pre-outlet channel 196 having a tenth distance 82 and an eleventh distance 71 may extend from the reaction chamber 70 to a residence time channel 80. A second flow 210 may exit the reaction chamber 70 through the pre-residence time channel 196 into the residence time channel 80.

The width of the reaction chamber 70 may be defined as a dimension orthogonal or parallel to a direction of flow of the inlet streams into the reaction chamber 70. In one embodiment, a diamond shaped geometry as illustrated in FIG. 3 may have a first aspect ratio, such that the first channel width 78 is orthogonal to the direction of flow. The first aspect ratio may be of at least 10, of at least 25, or of at least 40. The first aspect ratio, where the first chamber width 78 is orthogonal to the direction of flow, may be up to 100, up to 85, or up to 70. The first aspect ratio, where the first chamber width 78 is orthogonal to the direction of flow, may be in a range of 10 to 100, 25 to 85, or in a range of 30 to 70. The diamond shaped geometry may have a second aspect ratio, where the second chamber width 76 is parallel to the direction of flow, the second aspect ratio may be at least 10, of at least 25, or of at least 40. The second aspect ratio, where the second chamber width 76 is parallel to the direction of flow, may be up to 100, up to 85, or up to 70. The second aspect ratio, where the second chamber width 76 is parallel to the direction of flow, may be in a range of 10 to 100, 25 to 95, or in a range of 50 to 90. The reaction chamber 70, having dimensions relative to the first aspect ratio and the second aspect ratio, may provide sufficient heat removal during formation of the reaction product. In one embodiment, the first aspect ratio is 54, and the second aspect ratio is 60. In some embodiments, the first aspect ratio may be greater than the second aspect ratio.

In some reaction chambers, the fluid flow of reactants flowing into a reaction chamber can be characterized by a Reynolds number less than 2100, and is laminar. In this regime, viscous effects can dominate over inertial effects, reducing or nearly eliminating convective mixing. Molecules typically diffuse and mix according to an Einstein relation for molecular diffusion in solution. Diffusive time scales are also typically longer than convective time scales, further contributing to poor diffusive mixing. Mechanical mixing can also be used to mix reactants at a microscale; however, viscous forces dominate and a large amount of energy is required to overcome these forces. Internal passive mixing is another method relying on passive means to mix components. Static mixers are another common solution for mixing, and can be incorporated into a reactor system, but add complexity and cost. Another method of mixing relies on the reactive properties of the reactants, and the reaction products and byproducts evolved.

A chemical reaction may occur when reactants of inlet streams contact one another in a reaction chamber. For example, when an α,β-unsaturated carboxyl acid reacts with a halogenating agent, a gas may be evolved. In some embodiments, the gas evolved from the chemical reaction may provide a method of mixing within a reaction chamber. The evolution of gas may aid in mass transfer between the inlet streams where laminar flow typically would prevent mixing between the two streams other than by diffusion. Chaotic mixing (e.g., chaotic advection) may be described as a means for mixing reactants without diffusive or mechanical means, such that a gas evolved during the chemical reaction mixes the reactants sufficient for forming a reaction product. Chaotic advection can be further described in Ottino, J. M., Annu. Rev. Fluid Mech., 22, 207-253 (1990).

In one embodiment, the reaction chamber 70 of FIG. 3 may have a first chamber width 78, and a second chamber width 76. The reaction chamber 70 may have sufficient volume to provide for mixing of a first inlet stream, a second inlet stream, and an optional third inlet stream. In one embodiment, the inlet streams may be mixed by chaotic mixing within the reaction chamber 70. In another embodiment, the reaction chamber 70 is free of static mixing or mechanical mixing means.

A flow-through microreactor may comprise a reaction stream. The reaction stream may flow into a residence time channel 80 of FIG. 2 from a reaction chamber 70. The reaction stream may move through the residence time channel 80 to an outlet 90. The reaction stream may exit the outlet 90. The reaction stream may comprise a reaction product, unreacted reactants, byproducts, or combinations thereof. In some embodiments, the reaction stream without purification comprises an α,β-unsaturated carbonyl halide, an optional Michael addition product, and other byproducts. In other embodiments, the reaction stream without purification comprises the α,β-unsaturated carbonyl halide, and no detectable amount of a Michael addition product or byproducts. Proton nuclear magnetic resonance (¹H NMR) spectroscopy is one example of an analytical technique that may be used for the determination of the reaction product, unreacted reactants, Michael addition products, and other byproducts.

A residence time channel 80 may be provided having a length such that a reaction stream comprising remaining reactants from a chemical reaction after exiting a reaction chamber 70 may continue to react for forming a reaction product. In one embodiment, the chemical reaction is solventless. The reaction product may form in the residence time channel 80 as the reaction stream flows toward the outlet 90 for achieving a higher percent yield of α,β-unsaturated carbonyl halides. The flow of the reaction stream may be controlled or regulated in part from the flow of the inlet streams into the reaction chamber 70, and the subsequent exit of the reaction stream from the reaction chamber 70 into the residence time channel 80. The outlet 90 may have a diameter in a range of 1 to 5 mm. In one embodiment, the residence time channel 80 may have a serpentine configuration. Other examples of residence time channels may include linear, circular, spherical, ellipsoidal, and combinations thereof. Any suitable residence channel length 80 can be used, such that the chemical reaction may continue to from reaction products after exiting the reaction chamber 70. In some embodiments, the length of the residence time channel 80 may be at least 20 cm, at least 40 cm, at least 60 cm, or at least 100 cm. The length of the residence time channel 80 may be up to 250 cm, up to 200 cm, up to 150 cm, or up to 125 cm. The length of the residence time channel 80 may be in a range of 20 to 250 cm, 40 to 200, 40 to 150 cm, or in a range of 60 to 250 cm.

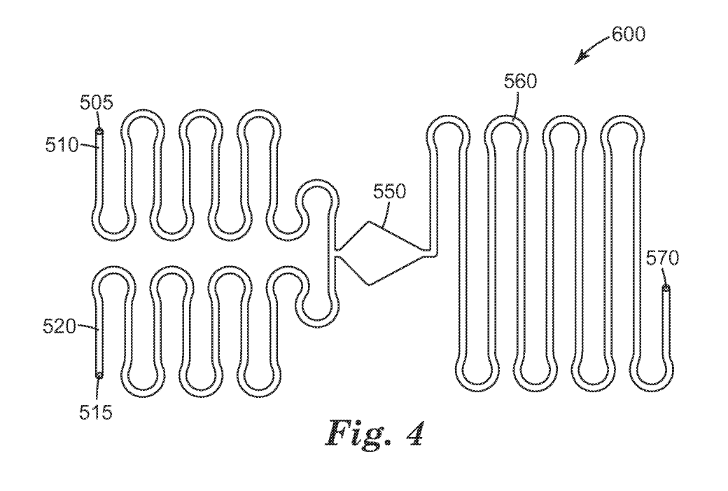

In another embodiment, FIG. 4 illustrates an exemplary flow-through microreactor 600 comprising serpentine inlet channels flowing into a diamond shaped reaction chamber. The flow-through microreactor 600 comprises a first inlet 505 for the addition of a first inlet stream, and a second inlet 515 for the addition of a second inlet stream. The first inlet 505 may be connected to a first inlet channel 510, and the second inlet 515 may be connected to a second inlet channel 520. The first inlet channel 510 and the second inlet channel 520 may each have a serpentine configuration. The first inlet channel 510 and the second inlet channel 520 may be heated or cooled. The first inlet stream can flow through the first inlet channel 510, and the second inlet stream can flow through the second inlet channel 520, such that the first inlet stream and the second inlet stream contact each other in a reaction chamber 550. The reaction chamber 550 may be heated or cooled. As the reactants contact one another for a chemical reaction, a reaction product may be formed. The reaction product of a reaction stream may flow through a residence time channel 560 to an outlet 570. The residence time channel 560 may have a serpentine configuration.

The disclosure will be further clarified by the following examples which are exemplary and not intended to limit the scope of the disclosure.

### EXAMPLES

The present disclosure is more particularly described in the following examples that are intended as illustrations only, since numerous modifications and variations within the scope of the present disclosure will be apparent to those skilled in the art. Unless otherwise noted, all parts, percentages, and ratios reported in the following examples are on a weight basis, and all reagents used in the examples were obtained, or are available, from the chemical suppliers described below, or may be synthesized by conventional techniques.

### Microreactor Description

A flow through microreactor having a volume of 0.4 ml was used for forming α,β-unsaturated carbonyl halides. The flow-through microreactor has at least two inlets for the addition of at least two inlet streams. The at least two inlet streams flow through the two inlet channels having a serpentine configuration into a reaction chamber. The reaction stream formed in the reaction chamber flows into a residence time channel having a serpentine configuration. After flowing through the residence time channel, the reaction stream flows through an outlet of the microreactor.

### Example 1

Acrylic acid (99%, Sigma Alrich, St. Louis, Mo.) was mixed with dimethylformamide ((DMF), Sigma Alrich, St. Louis, Mo.) as a catalyst to form a first inlet stream. Two mole percent DMF based on the moles of acrylic acid was added to the acrylic acid. Oxalyl chloride (98%, Alfa-Aesar, Ward Hill, Mass.) was used as a second inlet stream. A first 20 ml syringe comprising the first inlet stream and a second 20 ml syringe comprising a second inlet stream were placed in a syringe pump assembly (Model #11 Plus, Harvard Apparatus, Holliston, Mass.). The syringes within the syringe pump assembly were attached with PTFE tubing to the flow-through microreactor. The first inlet stream was delivered to a first inlet, and the second inlet stream was delivered to a second inlet at room temperature. The reaction chamber was set at a temperature of 40° C. The first inlet stream was added to the first inlet at a flow rate of 0.050 ml/minute. The second inlet stream was added to the second inlet at a flow rate of 0.068 ml/minute. A 10 percent molar excess of the oxalyl chloride relative to the acrylic acid was added via the first and second inlets to achieve a molar stoichiometry of 1.1:1. The first inlet stream flowed through a first serpentine inlet channel, and the second inlet stream flowed through a second serpentine inlet channel. The first inlet stream and the second inlet then flowed into a reaction chamber. Once the reactants contacted one another, gases such as hydrochloric acid (HCl), carbon dioxide (CO₂), and carbon monoxide (CO) were generated during the solventless chemical reaction. The gases evolved within the reaction chamber mixed the reactants to form a reaction product. The reaction stream comprising the reaction product, and optional byproducts was flowed from the reaction chamber through a residence time channel to an outlet. A sample of the reaction stream was recovered from the outlet at about 10 minutes from the initial addition of the first inlet stream and the second inlet stream into the flow-through microreactor.

The reaction stream was recovered from the outlet, and a sample was collected in a 5 mm nuclear magnetic resonance (NMR) tube (Wilmad LabGlass, Buena, N.J.). A small volume of CDCl₃ (99.8% purity+1% TMS, Cambridge Isotope Labs, Andover, Mass.) was added to the sample in the NMR tube as the NMR solvent. The reaction stream was analyzed via ¹H NMR (Varian Inova 400 MHz, Varian Inc., Palo Alto, Calif.). Sixteen scans were conducted to develop the ¹H NMR spectrum. Integration of the proton peaks indicated approximately 96.2 mol % yield of acryloyl chloride: 6.64 (1H, d, J=16 Hz), 6.35 (1H, dd, J=16 Hz, J=8 Hz), 6.18 (1H, d, J=8 Hz); 2.1 mol % of the product resulting from the addition of HCl to the olefin: 3.77 (2H, t, J=6.5 Hz), 3.35 (2H, t, J=6.4 Hz); and 1.7 mol % of the Michael addition product: 6.44 (1H, dd, J=17.4, 1.4 Hz), 6.12 (1H, dd, J=17.3, 10.5 Hz), 5.88 (1H, dd, J=10.4, 1.4 Hz), 4.46 (2H, t, J=5.9 Hz), 3.26 (2H, t, J=6 Hz). Other unidentified impurities were observed in the 6-7 ppm range. DMF catalyst was also observed in the product mixture.

### Example 2

Acrylic acid (99%, Sigma Alrich, St. Louis, Mo.) was used as a first inlet stream. Oxalyl chloride (98%, Alfa-Aesar, Ward Hill, Mass.) was used as a second inlet stream. A first 20 ml syringe comprising the first inlet stream and a second 20 ml syringe comprising a second inlet syringe were placed in a syringe pump assembly (model #11 Plus, Harvard Apparatus, Holliston, Mass.). The syringes within the syringe pump assembly were attached with PTFE tubing to the flow through microreactor. The first inlet stream was delivered to a first inlet, and the second inlet stream was delivered to a second inlet of the flow through microreactor at room temperature. The reaction chamber was set at a temperature of 40° C. The first inlet stream was added to the first inlet at a flow rate of 0.050 ml/minute. The second inlet stream was added to the second inlet at a flow rate of 0.068 ml/minute. A 10 percent molar excess of the oxalyl chloride relative to the acrylic acid was added via the first and second inlet ports to achieve a molar stoichiometry of 1.1:1. The first inlet stream flowed through a first serpentine inlet channel, and the second inlet stream flowed through a second serpentine inlet channel. The first inlet stream and the second inlet then flowed into a reaction chamber. Once the reactants contacted one another, gases such as hydrochloric acid (HCl), carbon dioxide (CO₂), and carbon monoxide (CO) were generated during the solventless chemical reaction. The gases evolved within the reaction chamber mixed the reactants to form a reaction product. The reaction stream comprising the reaction product, and optional byproducts was flowed from the reaction chamber through a residence time channel to an outlet. A sample of the reaction stream was recovered from the outlet at about 10 minutes from the initial addition of the first inlet stream and the second inlet stream to the flow-through microreactor.

The reaction stream was recovered from the outlet, and a sample was collected in a 5 mm nuclear magnetic resonance (NMR) tube (Wilmad LabGlass, Buena, N.J.). A small volume of CDCl₃ (99.8% purity+1% TMS, Cambridge Isotope Labs, Andover, Mass.) was added to the sample in the NMR tube as the NMR solvent. The reaction stream was analyzed via ¹H NMR (Varian Inova 400 MHz, Varian Inc., Palo Alto, Calif.). Sixteen scans were conducted to develop the ¹H NMR spectrum. Integration of the proton peaks indicated approximately 5.7 mol % yield of acryloyl chloride: 6.64 (1H, d, J=16 Hz), 6.35 (1H, dd, J=16, 8 Hz), 6.18 (1H, d, J=8 Hz), 94.3 mol % of unreacted acrylic acid: 12.2 (1H, s), 6.53 (1H, dd, J=17.2, 1.2 Hz), 6.15 (1H, dd, J=17.2, 10.4 Hz), 5.97 (1H, dd, J=10.4, 1.2 Hz). Other unidentified impurities were observed in the 6-7 ppm range.

### Example 3

Methacrylic acid (99%, Alfa-Aesar, Ward Hill, Mass.) was mixed with dimethylformamide ((DMF), Sigma Alrich, St. Louis, Mo.) as a catalyst to form a first inlet stream. Two mole percent DMF based on the moles of methacrylic acid was added to the methacrylic acid. Oxalyl chloride (98%, Alfa-Aesar, Ward Hill, Mass.) was used as a second inlet stream. A first 20 ml syringe comprising the first inlet stream and a second 20 ml syringe comprising a second inlet stream were placed in a syringe pump assembly (model #11 Plus, Harvard Apparatus, Holliston, Mass.). The syringes within the syringe pump assembly were attached with PTFE tubing to the flow-through microreactor. The first inlet stream was delivered to a first inlet, and the second inlet stream was delivered to a second inlet at room temperature. The reaction chamber was set at a temperature of 60° C. The first inlet stream was added to the first inlet at a flow rate of 0.040 ml/minute. The second inlet stream was added to the second inlet at a flow rate of 0.044 ml/minute. A 10 percent molar excess of the oxalyl chloride relative to the methacrylic acid was added via the first and second inlets to achieve a molar stoichiometry of 1.1:1. The first inlet stream flowed through a first serpentine inlet channel, and the second inlet stream flowed through a second serpentine inlet channel. The first inlet stream and the second inlet then flowed into a reaction chamber. Once the reactants contacted one another, gases such as hydrochloric acid (HCl), carbon dioxide (CO₂), and carbon monoxide (CO) were generated during the solventless chemical reaction. The gases evolved within the reaction chamber mixed the reactants to form a reaction product. The reaction stream comprising the reaction product, and optional byproducts was flowed from the reaction chamber through a residence time channel to an outlet. A sample of the reaction stream was recovered from the outlet at about 10 minutes from the initial addition of the first inlet stream and the second inlet stream to the flow-through microreactor.

The reaction stream was recovered from the outlet, and a sample was collected in a 5 mm nuclear magnetic resonance (NMR) tube (Wilmad LabGlass, Buena, N.J.). A small volume of CDCl₃ (99.8% purity+1% TMS, Cambridge Isotope Labs, Andover, Mass.) was added to the sample in the NMR tube as the NMR solvent. The reaction stream was analyzed via ¹H NMR (Varian Inova 400 MHz, Varian Inc., Palo Alto, Calif.). Sixteen scans were conducted to develop the ¹H NMR spectrum. Integration of the proton peaks indicated approximately 99+mol % yield of methacryloyl chloride: 6.51 (1H, m), 6.03 (1H, m), 2.02 (3H, m).

### Example 4

Methacrylic acid (99%, Alfa-Aesar, Ward Hill, Mass.) was mixed with dimethylformamide ((DMF), Sigma Alrich, St. Louis, Mo.) as a catalyst to form a first inlet stream. Two mole percent DMF based on the moles of methacrylic acid was added to the methacrylic acid. Thionyl chloride (99%, Alfa-Aesar, Ward Hill, Mass.) was used as a second inlet stream. A first 20 ml syringe comprising the first inlet stream and a second 20 ml syringe comprising a second inlet stream were placed in a syringe pump assembly (model #11 Plus, Harvard Apparatus, Holliston, Mass.). The syringes within the syringe pump assembly were attached with PTFE tubing to the flow through microreactor. The first inlet stream was delivered to a first inlet, and the second inlet stream was delivered to a second inlet of the flow-through microreactor at room temperature. The reaction chamber was set at a temperature of 60° C. The first inlet stream was added to the first inlet at a flow rate of 0.040 ml/minute. The second inlet stream was added to the second inlet at a flow rate of 0.038 ml/minute. A 10 percent molar excess of the thionyl chloride relative to the methacrylic acid was added via the first and second inlet ports to achieve a molar stoichiometry of 1.1:1. The first inlet stream flowed through a first serpentine inlet channel, and the second inlet stream flowed through a second serpentine inlet channel. The first inlet stream and the second inlet stream then flowed into a reaction chamber. Once the reactants contacted one another, hydrochloric acid (HCl) gas was generated during the solventless chemical reaction. The gas evolution within the reaction chamber mixed the reactants to form a reaction product. The reaction stream comprising the reaction product, and optional byproducts was flowed from the reaction chamber through a residence time channel to an outlet. A sample of the reaction stream was recovered from the outlet at about 10 minutes from the initial addition of the first inlet stream and the second inlet stream to the flow-through microreactor.

The reaction stream was recovered from the outlet, and a sample was collected in a 5 mm nuclear magnetic resonance (NMR) tube (Wilmad LabGlass, Buena, N.J.). A small volume of CDCl₃ (99.8% purity+1% TMS, Cambridge Isotope Labs, Andover, Mass.) was added to the sample in the NMR tube as the NMR solvent. The reaction stream was analyzed via ¹H NMR (Varian Inova 400 MHz, Varian Inc., Palo Alto, Calif.). Sixteen scans were conducted to develop the ¹H NMR spectrum. Integration of the proton peaks indicated approximately 75 mol % yield of acryloyl chloride: 6.64 (1H, d, J=16 Hz), 6.35 (1H, dd, J=16 Hz, J=8 Hz), 6.18 (1H, d, J=8 Hz); 10.5 mol % of the product resulting from the addition of HCl to the olefin: 3.77 (2H, t, J=6.5 Hz), 3.35 (2H, t, J=6.4 Hz); 14.5% of the Michael addition product: 6.44 (1H, dd, J=17.4, 1.4 Hz), 6.12 (1H, dd, J=17.3, 10.5 Hz), 5.88 (1H, dd, J=10.4, 1.4 Hz), 4.46 (2H, t, J=5.9 Hz), 3.26 (2H, t, J=6 Hz). DMF catalyst was also observed in the product mixture.

Various modifications and alterations of this disclosure will be apparent to those skilled in the art without departing from the scope and spirit of this disclosure, and it should be understood that this disclosure is not limited to the illustrative elements set forth herein.

What is claimed is:

1. A process for forming an α,β-unsaturated carbonyl halide, the process comprising:
providing reactants for a solventless reaction, the reactants comprising
(a) an α,β-unsaturated carboxylic acid in a first inlet stream;
(b) a halogenating agent in a second inlet stream; and
(c) a catalyst in the first inlet stream or in an optional third inlet stream; and
flowing the first inlet stream, the second inlet stream, and the optional third inlet stream into a reaction chamber of a flow-through microreactor; and
forming a reaction product comprising an α,β-unsaturated carbonyl halide.

2. The process of claim 1, wherein a temperature of the first inlet stream, the second inlet stream, and the optional third inlet stream independently flowing into the reaction chamber is in a range of −20° C. to 100° C.

3. The process of claim 1, wherein a temperature of the reactants in the reaction chamber is in a range of −20° C. to 100° C.

4. The process of claim 1, wherein the α,β-unsaturated carboxylic acid is of the formula: 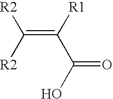
wherein
R1 comprises hydrogen, substituted alkyl, unsubstituted alkyl, aryl, substituted aryl, or combinations thereof; and
each R2 independently comprises hydrogen, substituted alkyl, unsubstituted alkyl, substituted aryl, unsubstituted aryl, carboxyl, or combinations thereof.

5. The process of claim 1, wherein the α,β-unsaturated carboxylic acid is acrylic acid or methacrylic acid.

6. The process of claim 1, wherein the halogenating agent comprises oxalyl chloride, thionyl chloride, phosgene, phosphorus trichloride, phosphorous tribromide, or combinations thereof.

7. The process of claim 1, wherein the halogenating agent entering the reaction chamber is a gas.

8. The process of claim 1, wherein the halogenating agent is present in a molar excess to the α,β-unsaturated carboxylic acid, the molar excess in a range of 0.01 to 10 percent.

9. The process of claim 1, wherein the α,β-unsaturated carboxylic acid is present in a molar excess to the halogenating agent, the molar excess in a range of 0.01 to 10 percent.

10. The process of claim 1, wherein the catalyst comprises dimethylformamide, pyridine, imidazole, or combinations thereof.

11. The process of claim 1, wherein the catalyst is in a range of 0.01 to 5 mole percent of the α,β-unsaturated carboxylic acid.

12. The process of claim 1, wherein a first aspect ratio of the reaction chamber in a direction perpendicular to a direction of flow is in a range of 30 to 70.

13. The process of claim 1, wherein a second aspect ratio of the reaction chamber in a direction parallel to a direction of flow is in a range of 50 to 90.

14. The process of claim 1, wherein the flow-through microreactor has a volume in a range of 0.01 to 20 milliliters.

15. The process of claim 1, wherein the forming of the reaction product evolves a gas, the gas providing chaotic mixing in the reaction chamber.

16. The process of claim 1, wherein the reaction chamber is free of static mixing or mechanical mixing means.

17. The process of claim 1, wherein the reaction product further comprises a Michael addition product, wherein the Michael addition product is less than 10 mole percent.

18. A process for forming an α,β-unsaturated carbonyl halide, the process comprising:
providing reactants comprising
(a) an α,β-unsaturated carboxylic acid in a first inlet stream;
(b) a halogenating agent comprising oxalyl chloride in a second inlet stream; and
(c) a catalyst in the first inlet stream or in an optional third inlet stream;
flowing the first inlet stream, the second inlet stream, and the optional third inlet stream into a reaction chamber of a flow-through microreactor; and
forming a reaction product comprising an α,β-unsaturated carbonyl halide, the α,β-unsaturated carbonyl halide having a percent yield of greater than 90 percent.

19. The process of claim 18, wherein at least one of the first inlet stream, the second inlet stream, or the optional third inlet stream comprises a solvent.

20. The process of claim 18, where the process is solventless.
